(19) United States
(12) Patent Application Publication
(10) Pub. No.: US 2024/0379194 A1
(43) Pub. Date: Nov. 14, 2024

(54) PATIENT FRICTION COEFFICIENT MODEL

(71) Applicant: IQVIA Inc., Parsippany, NJ (US)

(72) Inventors: Clifford David Cameron, Pittsboro, NC (US); Denise Messer, Apex, NC (US); Michal Galusza, Kwirynów (PL); Cara Willoughby, Chapel Hill, NC (US)

(21) Appl. No.: 18/315,182

(22) Filed: May 10, 2023

(51) Int. Cl. G16H 10/20; G16H 10/60

(52) U.S. Cl. CPC G16H 10/20; G16H 10/60

(57) ABSTRACT

A method includes determining, for one or more variables that describe a potential subject for a study, corresponding functions representing a relationship between the one or more variables and (i) an estimated burden or (ii) an estimated burden reduction that would be imposed on the potential subject by a protocol of the study if the potential subject were to participate in the study. The estimated burden or the estimated burden reduction is dependent on subject-specific values for the one or more variables absent participation of the potential subject in the study. The method includes using the corresponding functions to determine, for each individual of a plurality of individuals, a set of estimated burden values or estimated burden reduction values associated with the one or more variables. The method also includes identifying a subset of the plurality of individuals or a patient profile to be prioritized for recruitment for the study.

### TECHNICAL FIELD

This specification relates to research studies such as clinical trials, and the recruitment of study subjects (also referred to as “study participants”) for said research studies.

### BACKGROUND

Clinical trials are research studies performed on study participants (e.g., human subjects) to assess one or more tests and/or interventions (e.g., medical, surgical, behavioral, etc.). Designing a protocol for a clinical trial can involve identifying a target study population, including identifying one or more eligibility criteria for potential subjects, and then recruiting and enrolling subjects from a pool of eligible potential subjects. The statistical power, and consequently the scientific value, of clinical trials can be highly dependent on the number of enrolled subjects, the number of enrolled subjects who are retained throughout the length of the study, the level of adherence of the enrolled subjects to the study protocol, the subjects' representativeness of a real world target population, etc. Therefore, designing a protocol for a successful clinical trial can involve careful consideration of who to recruit into the clinical trial.

### SUMMARY

This document describes techniques for analyzing data from one or more sources to derive models of the relationships between certain variables and the burden likely to be experienced by a subject participating in a particular study. This document further describes computational approaches that utilize the modelled relationships to identify, from a group of potential subjects, a subset of the potential subjects who are likely to be least burdened overall by participation in a particular study (e.g., a clinical trial). For example, the group of potential subjects may be a group of potential human subjects who satisfy one or more eligibility criteria for a study, and the subset of the potential subjects may be a subset of the eligible potential subjects who are then prioritized for recruitment into the study. In some implementations, rather than identifying a subset of particular individuals to prioritize for recruitment into the study, the techniques described herein can be utilized to identify a least burdened “patient profile” so that patients who match the patient profile can later be identified and prioritized for recruitment. Prioritizing recruitment of those individuals who are likely to be least burdened by participation in the study can increase the likelihood that the participants in the clinical trial satisfy one or more outcomes with respect to the study (e.g., enrollment in the study, retention in the study, completion of the study, adherence to a protocol of the study, etc.). While many of the examples described herein describe clinical trials in which the potential subjects are “patients” (e.g., human individuals being treated for a disease), the techniques described in this document can be readily extended to potential subjects for all types and phases of clinical studies (e.g., vaccine/prevention studies, pharmacokinetics and pharmacodynamics studies, real-world evidence studies, etc.) including healthy volunteers, non-human animal subjects, etc.

The techniques described herein for identifying a “least burdened subset” of subjects incorporates a potential subject's current experience (e.g., absent participation in the study) to estimate the incremental burden or reduction in burden that would be imposed on the potential subject if they were to participate in the study. For example, compared to an individual who does not currently make regular hospital visits at all, a patient who already visits a hospital twice a month due to a medical condition is likely to be less burdened by participating in a study that requires three visits to the hospital per month. Similarly, compared to an individual who lives 50 miles away from a clinical trial site, a potential subject who lives 2 miles away from the clinical trial is likely to be less burdened by participating in a study that requires travelling to the clinical trial site. To estimate these (and other) effects on patient burden, the technology described herein includes data-driven approaches for determining one or more “feature functions” that model the effect of one or more variables (sometimes referred to herein as “features”) on the burden experienced by a potential subject. These variables can include indicators of a visit schedule, indicators of disease (e.g., severity of disease, number of years of treatment, etc.), age, gender, travel distance to a trial location, etc. By combining longitudinal data about potential subjects (e.g., electronic health record data, electronic medical record data, etc.), questionnaire data from potential subjects, information about a study protocol, and data from other sources, the technology described herein enables the improved identification of a least burdened subset of individuals who can then be prioritized for recruitment into a study.

Various implementations of the technology described herein may provide one or more of the following advantages. Compared to existing approaches that do not consider the burdens on an eligible potential subject until they are screened, the technology described herein can utilize various sources of data (e.g., longitudinal patient data, questionnaire data, published studies, input from subject matter experts, etc.) to estimate the incremental burden (or burden reduction) likely to be incurred by a potential subject much earlier in the subject selection process. This can save substantial time, money, and effort by enabling more targeted recruiting of potential subjects for a study.

Furthermore, compared to existing approaches that consider a study protocol's complexity and target the protocol to a particular trial site (e.g., based on the site's medical rating, specialization, past performance, available patient population, etc.), the technology described herein has the advantage of considering the patient perspective.

Another advantage of the technology described herein is that the feature functions developed to estimate patient burden can be externally validated and applied across various study protocols, thereby enabling direct comparison of burden between different study protocols.

Yet another advantage of the technology described herein is that the feature functions and their relative weightings (e.g., when estimating an overall burden metric) can be based, at least partially, on electronic medical records data rather than survey or questionnaire data alone. Compared to existing methodologies that evaluate study protocols solely based on survey or questionnaire data, the incorporation of longitudinal data (e.g., electronic medical records data) can yield less biased results and more nuanced models of the effects of particular variables on patient burden. For example, individuals may find it difficult to express, in self-reported questionnaire data, that the number of visits they make to the hospital has an exponential effect on their perceived burden. However, an analysis of the distribution of hospital visits in a representative population of subjects (e.g., as determined from electronic medical records data) may reveal this exponential relationship more clearly.

In one aspect, a method is featured. The method includes determining, for one or more variables that describe a potential subject for a study, corresponding functions representing a relationship between the one or more variables and (i) an estimated burden or (ii) an estimated burden reduction that would be imposed on the potential subject by a protocol of the study if the potential subject were to participate in the study. The estimated burden or the estimated burden reduction is dependent on subject-specific values for the one or more variables absent participation of the potential subject in the study. The method also includes using the corresponding functions to determine, for each individual of a plurality of individuals, a set of estimated burden values or estimated burden reduction values associated with the one or more variables. The method further includes identifying, based on the set of estimated burden values or estimated burden reduction values for each individual of the plurality of individuals, a subset of the plurality of individuals or a patient profile to be prioritized for recruitment for the study.

Implementations can include the examples described below and herein elsewhere. In some implementations, the one or more variables include at least one of a visit schedule, an indicator of disease, an age, a gender, and a travel distance to a trial location. In some implementations, for each individual of the plurality of individuals, the subject-specific values for the one or more variables are determined, at least in part, based on questionnaire data or electronic medical records (EMR) data. In some implementations, for each individual of the plurality of individuals, at least some of the subject-specific values for the one or more variables represent historical values for the individual. In some implementations, for each individual of the plurality of individuals, at least some of the subject-specific values for the one or more variables represent predicted future values for the individual. In some implementations, the estimated burden is inversely related to a likelihood that the potential subject satisfies a particular outcome with respect to the study. In some implementations, the particular outcome can include at least one of enrollment in the study, retention in the study, completion of the study, or adherence to a protocol of the study. In some implementations, the corresponding functions can be convex and monotonically increasing. In some implementations, determining the corresponding functions can include determining a mathematical form of each of the corresponding functions based on distributions of the one or more variables in a population. In some implementations, determining the corresponding functions can include determining one or more parameters of the corresponding functions based on at least one of questionnaire data, electronic medical records (EMR) data, or subject matter expertise. In some implementations, determining the corresponding functions can include defining at least some of the corresponding functions such that the estimated burden is zero when the subject-specific values for the one or more variables absent participation of the potential subject in the study match values for the one or more variables specified by the protocol of the study. In some implementations, identifying the subset of the plurality of individuals or the patent profile to be prioritized for recruitment for the study can include computing, for each individual of the plurality of individuals, an aggregate metric representative of the set of estimated burden values; and identifying whether each individual of the plurality of individuals is below, above, or equal to a threshold value of the aggregate metric. In some implementations, the aggregate metric can be externally validated. In some implementations, the aggregate metric can be indicative of at least one of (i) a magnitude of a vector defined, at least in part, by the set of estimated burdens values or estimated burden reduction values, or (ii) a weighted combination of the set of estimated burden values or estimated burden reduction values. In some implementations, one or more weights of the weighted combination can be determined based on survey data reflective of one or more preferences of a population with respect to the one or more variables. In some implementations, identifying the subset of the plurality of individuals or the patient profile to be prioritized for recruitment for the study can include: computing, for each individual of the plurality of individuals, an aggregate metric representative of the set of estimated burden values or estimated burden reduction values; and identifying a specified number of the plurality of individuals or a specified percentage of the plurality of individuals based on a ranking of the plurality of individuals according to the aggregate metric. In some implementations, the method can include recruiting the identified individuals, or individuals that fit the patient profile, for the study. In some implementations, the method can include treating at least a portion of the identified individuals or individuals that fit the patient profile in accordance with the protocol of the study. In some implementations, the method can include determining that the identified subset of the plurality of individuals to be prioritized for recruitment for the study is below a threshold size, and altering the protocol of the study with respect to at least some of the one or more variables to increase a size of the identified subset of the plurality of individuals to be prioritized for recruitment for the study. In some implementations, the method can include comparing the protocol of the study with at least one other study protocol. Comparing the protocol of the study with the at least one other study protocol can include comparing the set of estimated burden values or estimated burden reduction values for each individual of the plurality of individuals with other burden-related calculations for the plurality of individuals, wherein the other burden-related calculations are associated with the at least one other study protocol.

In another aspect, a system is featured. The system includes a computing device that includes a memory configured to store instructions, and a processor configured to execute the instructions to perform operations. The operations include determining, for one or more variables that describe a potential subject for a study, corresponding functions representing a relationship between the one or more variables and (i) an estimated burden or (ii) an estimated burden reduction that would be imposed on the potential subject by a protocol of the study if the potential subject were to participate in the study. The estimated burden or the estimated burden reduction is dependent on subject-specific values for the one or more variables absent participation of the potential subject in the study. The operations also include using the corresponding functions to determine, for each individual of a plurality of individuals, a set of estimated burden values or estimated burden reduction values associated with the one or more variables. The operations further include identifying, based on the set of estimated burden values or estimated burden reduction values for each individual of the plurality of individuals, a subset of the plurality of individuals or a patient profile to be prioritized for recruitment for the study.

Implementations can include the examples described below and herein elsewhere. In some implementations, the one or more variables include at least one of a visit schedule, an indicator of disease, an age, a gender, and a travel distance to a trial location. In some implementations, for each individual of the plurality of individuals, the subject-specific values for the one or more variables are determined, at least in part, based on questionnaire data or electronic medical records (EMR) data. In some implementations, for each individual of the plurality of individuals, at least some of the subject-specific values for the one or more variables represent historical values for the individual. In some implementations, for each individual of the plurality of individuals, at least some of the subject-specific values for the one or more variables represent predicted future values for the individual. In some implementations, the estimated burden is inversely related to a likelihood that the potential subject satisfies a particular outcome with respect to the study. In some implementations, the particular outcome can include at least one of enrollment in the study, retention in the study, completion of the study, or adherence to a protocol of the study. In some implementations, the corresponding functions can be convex and monotonically increasing. In some implementations, determining the corresponding functions can include determining a mathematical form of each of the corresponding functions based on distributions of the one or more variables in a population. In some implementations, determining the corresponding functions can include determining one or more parameters of the corresponding functions based on at least one of questionnaire data, electronic medical records (EMR) data, or subject matter expertise. In some implementations, determining the corresponding functions can include defining at least some of the corresponding functions such that the estimated burden is zero when the subject-specific values for the one or more variables absent participation of the potential subject in the study match values for the one or more variables specified by the protocol of the study. In some implementations, identifying the subset of the plurality of individuals or the patent profile to be prioritized for recruitment for the study can include computing, for each individual of the plurality of individuals, an aggregate metric representative of the set of estimated burden values; and identifying whether each individual of the plurality of individuals is below, above, or equal to a threshold value of the aggregate metric. In some implementations, the aggregate metric can be externally validated. In some implementations, the aggregate metric can be indicative of at least one of (i) a magnitude of a vector defined, at least in part, by the set of estimated burdens values or estimated burden reduction values, or (ii) a weighted combination of the set of estimated burden values or estimated burden reduction values. In some implementations, one or more weights of the weighted combination can be determined based on survey data reflective of one or more preferences of a population with respect to the one or more variables. In some implementations, identifying the subset of the plurality of individuals or the patient profile to be prioritized for recruitment for the study can include: computing, for each individual of the plurality of individuals, an aggregate metric representative of the set of estimated burden values or estimated burden reduction values; and identifying a specified number of the plurality of individuals or a specified percentage of the plurality of individuals based on a ranking of the plurality of individuals according to the aggregate metric. In some implementations, the operations can include recruiting the identified individuals, or individuals that fit the patient profile, for the study. In some implementations, the operations can include treating at least a portion of the identified individuals or individuals that fit the patient profile in accordance with the protocol of the study. In some implementations, the operations can include determining that the identified subset of the plurality of individuals to be prioritized for recruitment for the study is below a threshold size, and altering the protocol of the study with respect to at least some of the one or more variables to increase a size of the identified subset of the plurality of individuals to be prioritized for recruitment for the study. In some implementations, the operations can include comparing the protocol of the study with at least one other study protocol. Comparing the protocol of the study with the at least one other study protocol can include comparing the set of estimated burden values or estimated burden reduction values for each individual of the plurality of individuals with other burden-related calculations for the plurality of individuals, wherein the other burden-related calculations are associated with the at least one other study protocol.

In another aspect, one or more machine-readable storage devices are featured. The one or more machine-readable storage devices have encoded thereon computer readable instructions for causing one or more processing devices to perform operations. The operations include determining, for one or more variables that describe a potential subject for a study, corresponding functions representing a relationship between the one or more variables and (i) an estimated burden or (ii) an estimated burden reduction that would be imposed on the potential subject by a protocol of the study if the potential subject were to participate in the study. The estimated burden or the estimated burden reduction is dependent on subject-specific values for the one or more variables absent participation of the potential subject in the study. The operations also include using the corresponding functions to determine, for each individual of a plurality of individuals, a set of estimated burden values or estimated burden reduction values associated with the one or more variables. The operations further include identifying, based on the set of estimated burden values or estimated burden reduction values for each individual of the plurality of individuals, a subset of the plurality of individuals or a patient profile to be prioritized for recruitment for the study.

Implementations can include the examples described below and herein elsewhere. In some implementations, the one or more variables include at least one of a visit schedule, an indicator of disease, an age, a gender, and a travel distance to a trial location. In some implementations, for each individual of the plurality of individuals, the subject-specific values for the one or more variables are determined, at least in part, based on questionnaire data or electronic medical records (EMR) data. In some implementations, for each individual of the plurality of individuals, at least some of the subject-specific values for the one or more variables represent historical values for the individual. In some implementations, for each individual of the plurality of individuals, at least some of the subject-specific values for the one or more variables represent predicted future values for the individual. In some implementations, the estimated burden is inversely related to a likelihood that the potential subject satisfies a particular outcome with respect to the study. In some implementations, the particular outcome can include at least one of enrollment in the study, retention in the study, completion of the study, or adherence to a protocol of the study. In some implementations, the corresponding functions can be convex and monotonically increasing. In some implementations, determining the corresponding functions can include determining a mathematical form of each of the corresponding functions based on distributions of the one or more variables in a population. In some implementations, determining the corresponding functions can include determining one or more parameters of the corresponding functions based on at least one of questionnaire data, electronic medical records (EMR) data, or subject matter expertise. In some implementations, determining the corresponding functions can include defining at least some of the corresponding functions such that the estimated burden is zero when the subject-specific values for the one or more variables absent participation of the potential subject in the study match values for the one or more variables specified by the protocol of the study. In some implementations, identifying the subset of the plurality of individuals or the patent profile to be prioritized for recruitment for the study can include computing, for each individual of the plurality of individuals, an aggregate metric representative of the set of estimated burden values; and identifying whether each individual of the plurality of individuals is below, above, or equal to a threshold value of the aggregate metric. In some implementations, the aggregate metric can be externally validated. In some implementations, the aggregate metric can be indicative of at least one of (i) a magnitude of a vector defined, at least in part, by the set of estimated burdens values or estimated burden reduction values, or (ii) a weighted combination of the set of estimated burden values or estimated burden reduction values. In some implementations, one or more weights of the weighted combination can be determined based on survey data reflective of one or more preferences of a population with respect to the one or more variables. In some implementations, identifying the subset of the plurality of individuals or the patient profile to be prioritized for recruitment for the study can include: computing, for each individual of the plurality of individuals, an aggregate metric representative of the set of estimated burden values or estimated burden reduction values; and identifying a specified number of the plurality of individuals or a specified percentage of the plurality of individuals based on a ranking of the plurality of individuals according to the aggregate metric. In some implementations, the operations can include recruiting the identified individuals, or individuals that fit the patient profile, for the study. In some implementations, the operations can include treating at least a portion of the identified individuals or individuals that fit the patient profile in accordance with the protocol of the study. In some implementations, the operations can include determining that the identified subset of the plurality of individuals to be prioritized for recruitment for the study is below a threshold size, and altering the protocol of the study with respect to at least some of the one or more variables to increase a size of the identified subset of the plurality of individuals to be prioritized for recruitment for the study. In some implementations, the operations can include comparing the protocol of the study with at least one other study protocol. Comparing the protocol of the study with the at least one other study protocol can include comparing the set of estimated burden values or estimated burden reduction values for each individual of the plurality of individuals with other burden-related calculations for the plurality of individuals, wherein the other burden-related calculations are associated with the at least one other study protocol.

In another aspect, another method is featured. The method includes using one or more functions to determine, for each individual of a plurality of individuals, a set of estimated burden values or estimated burden reduction values that would be imposed on the individual by a protocol of a study if the individual were to participate in the study. The one or more functions correspond to one or more variables that describe the individual, and the set of estimated burden values or estimated burden reduction values is dependent on individual-specific values for the one or more variables absent participation of the individual in the study. The method also includes identifying, based on the set of estimated burden values or estimated burden reduction values for each individual of the plurality of individuals, a subset of the plurality of individuals or a patient profile to be prioritized for recruitment for the study.

Implementations can include the examples described below and herein elsewhere. In some implementations, the one or more functions can be pre-defined by a third party. In some implementations, the one or more variables include at least one of a visit schedule, an indicator of disease, an age, a gender, and a travel distance to a trial location. In some implementations, for each individual of the plurality of individuals, the individual-specific values for the one or more variables are determined, at least in part, based on questionnaire data or electronic medical records (EMR) data. In some implementations, for each individual of the plurality of individuals, at least some of the individual-specific values for the one or more variables represent historical values for the individual. In some implementations, for each individual of the plurality of individuals, at least some of the individual-specific values for the one or more variables represent predicted future values for the individual. In some implementations, the estimated burden values for each individual of the plurality of individuals are inversely related to a likelihood that the individual satisfies a particular outcome with respect to the study. In some implementations, the particular outcome can include at least one of enrollment in the study, retention in the study, completion of the study, or adherence to a protocol of the study. In some implementations, the corresponding functions can be convex and monotonically increasing. In some implementations, the corresponding functions can be determined using a process that includes determining a mathematical form of each of the corresponding functions based on distributions of the one or more variables in a population. In some implementations, the corresponding functions can be determined using a process that includes determining one or more parameters of the corresponding functions based on at least one of questionnaire data, electronic medical records (EMR) data, or subject matter expertise. In some implementations, the corresponding functions can be determined using a process that includes defining at least some of the corresponding functions such that the estimated burden is zero when the subject-specific values for the one or more variables absent participation of the potential subject in the study match values for the one or more variables specified by the protocol of the study. In some implementations, identifying the subset of the plurality of individuals or the patent profile to be prioritized for recruitment for the study can include computing, for each individual of the plurality of individuals, an aggregate metric representative of the set of estimated burden values; and identifying whether each individual of the plurality of individuals is below, above, or equal to a threshold value of the aggregate metric. In some implementations, the aggregate metric can be externally validated. In some implementations, the aggregate metric can be indicative of at least one of (i) a magnitude of a vector defined, at least in part, by the set of estimated burdens values or estimated burden reduction values, or (ii) a weighted combination of the set of estimated burden values or estimated burden reduction values. In some implementations, one or more weights of the weighted combination can be determined based on survey data reflective of one or more preferences of a population with respect to the one or more variables. In some implementations, identifying the subset of the plurality of individuals or the patient profile to be prioritized for recruitment for the study can include: computing, for each individual of the plurality of individuals, an aggregate metric representative of the set of estimated burden values or estimated burden reduction values; and identifying a specified number of the plurality of individuals or a specified percentage of the plurality of individuals based on a ranking of the plurality of individuals according to the aggregate metric. In some implementations, the method can include recruiting the identified individuals, or individuals that fit the patient profile, for the study. In some implementations, the method can include treating at least a portion of the identified individuals or individuals that fit the patient profile in accordance with the protocol of the study. In some implementations, the method can include determining that the identified subset of the plurality of individuals to be prioritized for recruitment for the study is below a threshold size, and altering the protocol of the study with respect to at least some of the one or more variables to increase a size of the identified subset of the plurality of individuals to be prioritized for recruitment for the study. In some implementations, the method can include comparing the protocol of the study with at least one other study protocol. Comparing the protocol of the study with the at least one other study protocol can include comparing the set of estimated burden values or estimated burden reduction values for each individual of the plurality of individuals with other burden-related calculations for the plurality of individuals, wherein the other burden-related calculations are associated with the at least one other study protocol.

Other features and advantages of the description will become apparent from the following description, and from the claims. Unless otherwise defined, the technical and scientific terms used herein have the same meaning as commonly understood by one of ordinary skill in the art to which this invention belongs.

### BRIEF DESCRIPTION OF THE DRAWINGS

FIG. 1 shows a flowchart of a process for identifying a subset of potential subjects for a study or a patient profile from a group of potential subjects.

FIG. 2 shows a flowchart of a process for defining feature functions.

FIGS. 3A-3C show various approaches for identifying a subset of potential subjects or a patient profile for a study from a group of potential subjects.

FIG. 4 is a graph showing a cumulative frequency distribution of a patient friction coefficient (PFC) value in a group of potential subjects.

FIG. 5 is a graph showing a distribution of visit schedules for a group of potential subjects.

FIG. 6 is a graph showing distributions of visit schedules for various subsets of a group of potential subjects.

FIG. 7 is a graph showing a distribution of number of years of treatment for a group of potential subjects.

FIG. 8 is a graph showing a distribution of travel levels for a group of potential subjects.

FIG. 9 is a graph showing distributions of distance from an “ideal point” for various subsets of a group of potential subjects.

FIGS. 10-11 are flowcharts of processes for identifying a subset of a plurality of individuals or a patient profile to be prioritized for recruitment for a study.

FIG. 12 is a diagram illustrating an example of a computing environment.

### DETAILED DESCRIPTION

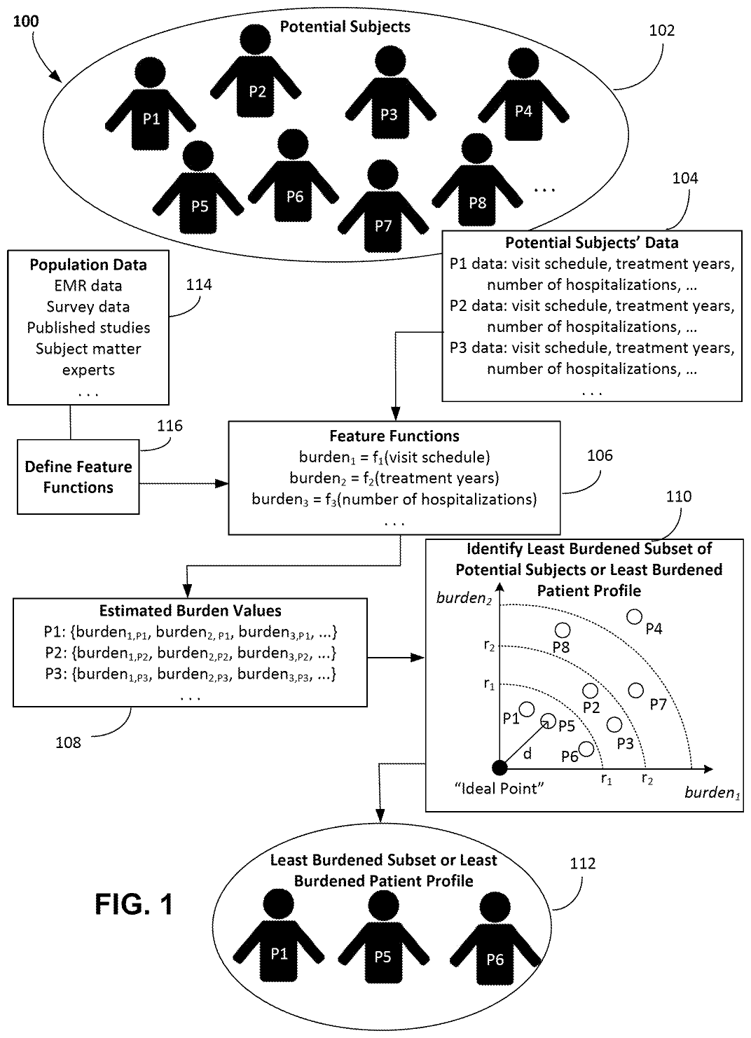

Referring to FIG. 1, a process 100 is depicted for identifying a least burdened subset of potential subjects or least burdened patient profile 112 for a study from a group of potential subjects 102. The group of potential subjects 102 includes a number of individuals (e.g., person P1, person P2, person P3, . . . , person P8), and the least burdened subset 112 includes a subset of those individuals (e.g., person P1, person P5, and person P6). In some cases, the group of potential subjects 102 can be representative of a population of interest. For example, the group of potential subjects 102 can comprise a group of individuals who satisfy the inclusion criteria for a particular study (e.g., having a particular condition, falling within a certain age range, not having a history of certain kinds of medical conditions or treatments, etc.). The potential subjects comprising the least burdened subset 112 can be those individuals from the group of potential subjects 102 who are identified as being likely to be least burdened by participation in the study (e.g., compared to a current experience of the individuals absent participation in the study). Since the estimated burdens of participation in the study are relatively low for the least burdened subset 112, the potential subjects comprising the least burdened subset 112 can, in turn, be the most likely to achieve or satisfy a particular outcome with respect to the study (e.g., enrollment in the study, retention in the study, completion of the study, adherence to a protocol of the study, etc.). Therefore, in some implementations, after identifying the least burdened subset 112, the individuals within the least burdened subset 112, or individuals similar to those in the least burdened subset (e.g., those sharing a similar patient profile), can be prioritized for recruitment into the study.

The process 100 for identifying the least burdened subset 112 begins with obtaining data 104 about a group of potential subjects 102 and obtaining population data 114. In some implementations, the group of potential subjects 102 can be anonymized and the obtained data 104 about the group can be anonymized. The obtained data 104 can include, for each individual in the group of potential subjects 102, information about a visit history of the individual to a healthcare facility (e.g., a frequency of hospital visits, a total number of visits made to a physician, etc.), an ongoing visit schedule of the individual to healthcare facilities, a number of years that the individual has received treatment for a particular condition, a number of times the individual has been hospitalized for a particular condition, any other indicator of an individual's disease condition, etc. The data 104 can also include demographic data about each individual in the group of potential subjects 102 including age, gender, relative location of home to clinical trial sites, etc. In some implementations, the data 104 can be obtained, for example, from longitudinal data sources such as electronic health records and electronic medical records. In some implementations, the data 104 can additionally (or alternatively) be obtained from questionnaires or surveys administered to the individuals in the group of potential subjects 102.

The population data 114 can come in a wide variety of different forms and can be obtained from a number of possible data sources. For example, the population data 114 can be obtained from longitudinal data sources such as electronic health records (EHR) or electronic medical records (EMR) data; from surveys administered to a representative sample of a relevant population; from published studies; from input given by subject matter experts; etc. In general, the population data 114 can include any information that may be useful for determining the relationship between one or more variables (e.g., indicators of disease, age, gender, distance from study site, etc.) and the burden likely to be experienced by a potential subject (or on a likelihood that the potential subject achieves a particular outcome with respect to the study). For example, the population data 114 may include EMR data representative of the distributions of one or more variables, such as a number of hospitalizations, within a relevant population. As another example, the population data 114 may include survey data in which a representative sample of a relevant population is asked what aspects of a study protocol are most influential on their willingness to participate. As yet another example, the population data 114 can include a published study demonstrating that women are more or less likely to adhere to certain study protocols compared to men.

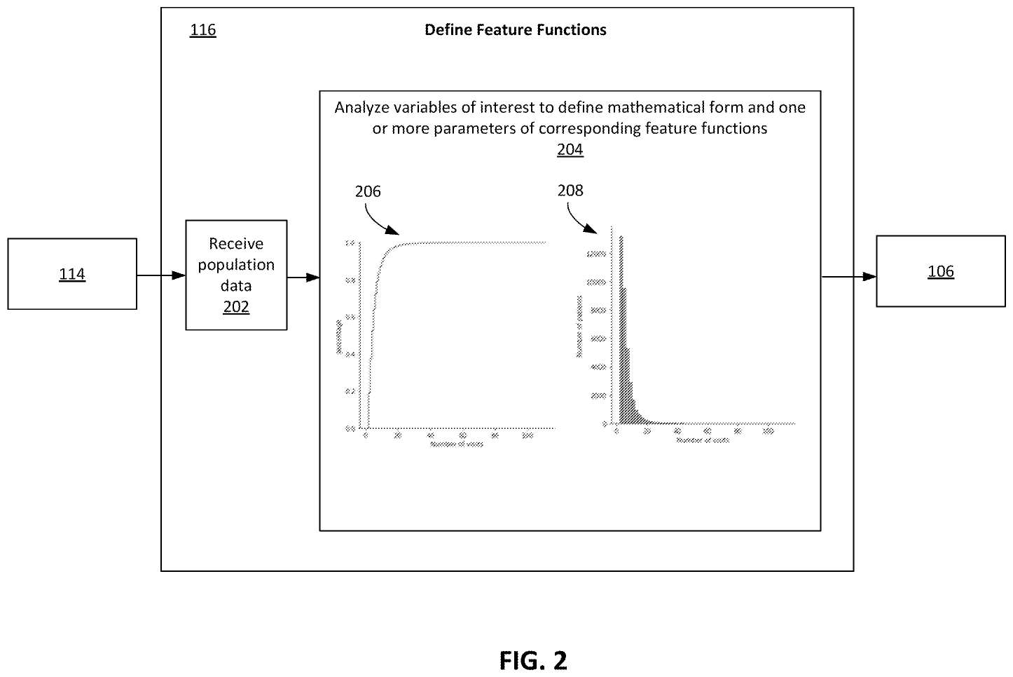

At step 116 of the process 100, the population data 114 is processed to define one or more feature functions. The term “feature function” is used throughout this document to refer to any mathematical function that models the relationship between one or more variables (or “features”) and the burden experienced by a potential subject. The term “burden”, in turn, is used broadly throughout this document, not only to refer to hardships or responsibilities that may be experienced by an individual, but any factor that may decrease the likelihood that the individual achieves or satisfies a particular outcome with respect to the study. For example, while being a man may not be a hardship or responsibility, it may still be considered a “burden” within the meaning of this document, if being a man correlates with a lower likelihood of adhering to a study protocol. A feature function corresponding to a “gender” variable may, therefore, include a mathematical function that models the relationship between gender and the likelihood of adhering to a study protocol. In some cases, at step 116, the one or more feature functions can be defined such that each feature function corresponds to a single variable (e.g., indicators of disease, age, gender, distance from study site, etc.). However, in some implementations, at least some of the one or more feature functions can be multivariable functions that model the relationship of multiple variables on the burden experienced by a potential subject. Techniques for defining feature functions are described in further detail below, for example, in relation to FIG. 2.

The output of step 116 of the process 100 is a set of defined feature functions 106. Each of the feature functions 106 takes, as input, values for one or more variables (e.g., indicators of disease, age, gender, distance from study site, etc.), and outputs an estimated burden value associated with the one or more variables. For example, referring to the example shown in FIG. 1, a first feature function can estimate a first burden value (“burden₁”) as a function of an individual's visit schedule to healthcare facilities. A second feature function can estimate a second burden value (“burden₂”) as a function of a number of years of treatment. A third feature function can estimate a third burden value (“burden₃”) as a function of a number of hospitalizations. Additional feature functions can estimate additional burden values associated with one or more additional variables such as age, gender, distance from study site, etc. In some implementations, the values for the one or more variables that are input to the feature functions 106 can be historical values for an individual (e.g., obtained from EMR data). However, in other implementations, the values for the one or more variables that are input to the feature functions 106 can be predicted future values for the individual (e.g., based on an expected prognosis for the individual if he/she does not participate in the study).

Using the feature functions 106 and the data 104, a set of estimated burden values 108 can be calculated for each of the individuals in the group of potential subjects 102. For example, the data 104 associated with individual P1 (e.g., visit schedule, treatment years, number of hospitalizations, etc.) can be input to the feature functions 106 to calculate a set of estimated burden values 108 associated with P1 (burden_{1,P1}, burden_{2,P1}, burden_{3,P1}, etc.). Data 104 associated with individual P2 can be input to the feature functions 106 to calculate a set of estimated burden values 108 associated with P2 (burden_{1,P2}, burden_{2,P2}, burden_{3,P2}, etc.). Similar calculations can be performed for each of the individuals in the group of potential subjects 102.

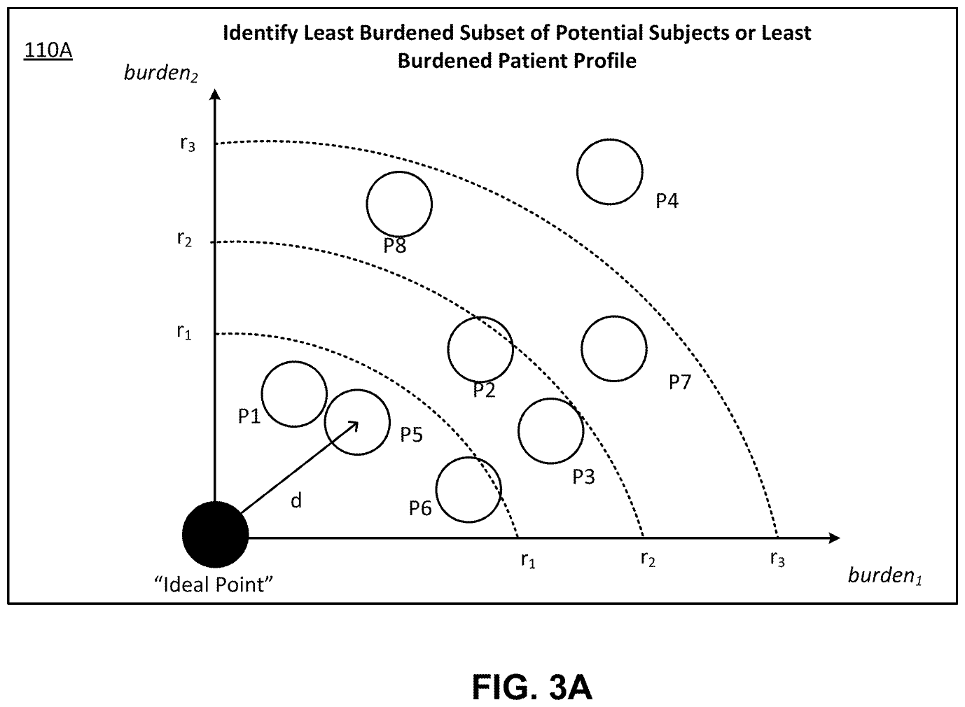

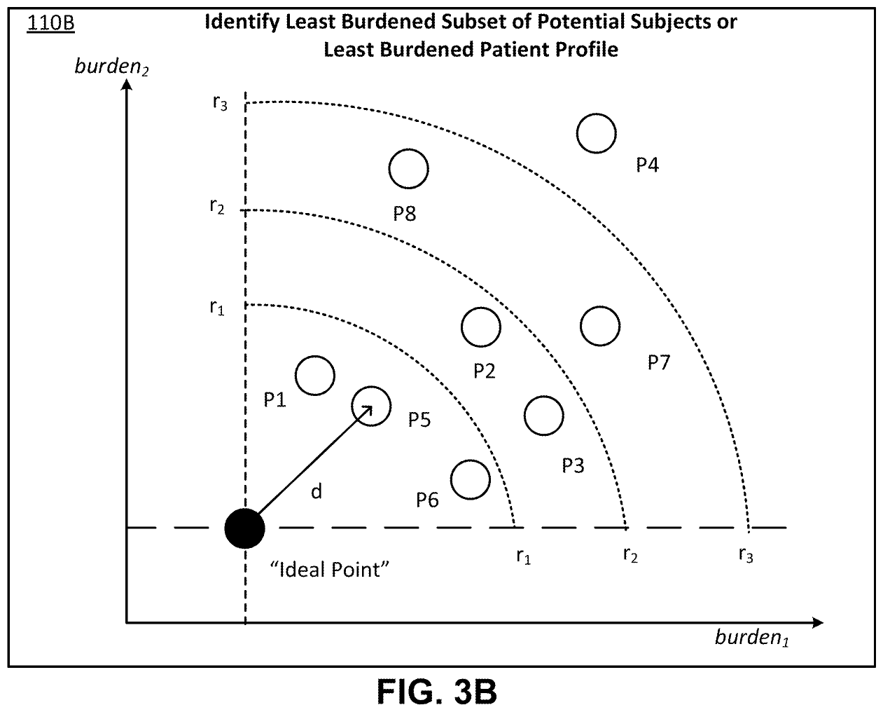

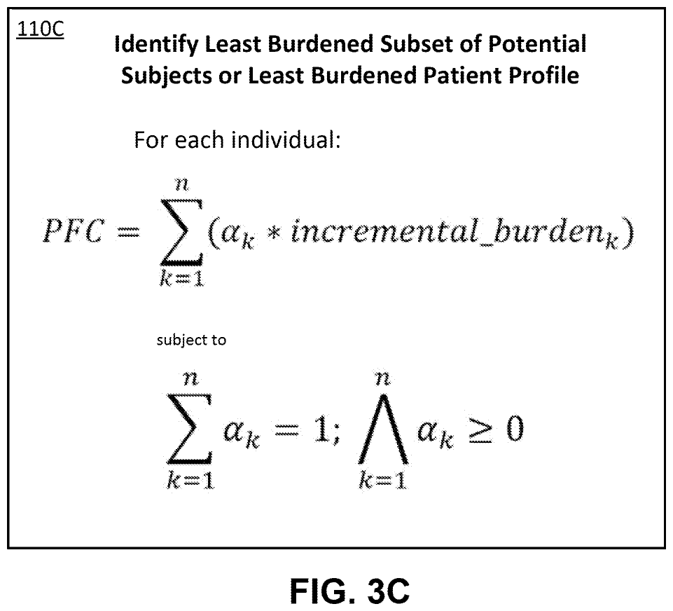

At step 110 of the process 100, the set of estimated burden values 108 associated with each individual in the group of potential subjects 102 can be used to identify the least burdened subset of potential subjects 112. Various approaches for the identification step 110 are described in further detail below, for example, with respect to FIGS. 3A-3C. However, generally, the step 110 involves comparing the estimated burden values for each individual to the estimated burden values associated with an “ideal point” to determine the estimated incremental burden (or estimated burden reduction) imposed on each individual compared to a hypothetical individual at the ideal point.

With respect to some variables, the ideal point may represent conditions that precisely reflect what is required by a study protocol such that a hypothetical individual at the ideal point would face no incremental burden (or burden reduction) by enrolling in the study. For example, if a study protocol requires three visits to the hospital per month and taking medication twice per day, a patient who already visits the hospital three times per month and takes medication twice per day would already be at the ideal point with respect to these variables. That is, enrolling in the study would not incur any incremental burden (or burden reduction) associated with these variables compared to the individual's current clinical care.

With respect to some variables, the ideal point may represent conditions that would minimize a burden associated with the study (or alternatively, maximize a likelihood of achieving or satisfying a particular outcome with respect to the study), even if a particular value for the variable is not required by the study protocol. For example, with respect to gender, even if a study is open to both men and women, the ideal point may be representative of a woman if women are more likely than men to adhere to a study protocol (and if adherence to the study protocol is the particular outcome of interest). This is because being a man can be interpreted as incurring an additional “burden” that may correlate with a decreased likelihood of adhering to/the study protocol.

In another example, with respect to a variable representing a number of hospitalizations, a study protocol may not require an individual to be hospitalized a certain number of times within a period of time prior to the study. However, a past history of hospitalizations may be indicative of increased disease severity, which is likely to lower the estimated incremental burden of participating in the study (e.g., since sicker individuals are more likely to be motivated to participate in the study in the hopes of getting better). Therefore, with respect to a number of hospitalizations, the ideal point may be representative of a hypothetical individual with a high number of previous hospitalizations.

In yet another example, with respect to a variable representing a distance from a study site, a study protocol may not require an individual to live within a certain distance from the study site. However, individuals who live closest to the study site are likely to have lower incremental burdens since enrolling in the study would result in less travel time compared to individuals who live farther away. Therefore, with respect to a variable representing a distance from a study site, the ideal point may be representative of a hypothetical individual who lives at the study site (e.g., having no incremental burden associated with traveling to the study site).

In general, every variable should be considered for bias and weighting in the model. Deciding whether or not to model the burden associated with a particular variable (or how much weight to give a particular variable) should balance the study objectives with the need to include a diverse population in the study and the importance of maintaining a representative study cohort.

Once the ideal point for the study has been determined, the estimated burdens associated with the ideal point are compared, at step 110 of the process 100, with the estimated burden values for each individual (e.g., individuals P1-P8) to identify the individuals who would incur the lowest estimated incremental burdens (or greatest estimated burden reductions) by enrolling in the study. These individuals (e.g., individuals P1, P5, P6) are identified as examples of the least burdened subset 112, and represent a patient profile that can be prioritized for recruitment into the study. For example, additional recruitment materials can be sent to the individuals P1, P5 and P6, or to other individuals who are similar to the least burdened subset 112 (e.g., individuals with a similar patient profile). Such individuals can be invited for further screening by researchers conducting the study. In some cases, if those who would fit the profile of the least burdened subset 112 of individuals is determined to be too small, or if the burden values for the least burdened subset 112 are unacceptably high, then the study protocol can be reviewed for potential modification to reduce the estimated incremental burdens associated with one or more individuals in the group of potential subjects 102. Alternatively or in addition, operational tactics can be implemented to mitigate or lower the incremental burdens associated with one or more individuals in the group of potential subjects 102 (e.g., incentivizing individuals by paying them, offering a drive service, offering child care, etc.). The advantage to conducting this burden modeling early allows the patient perspective to influence the protocol during design rather than requiring amendment when recruitment and retention are found to be low.

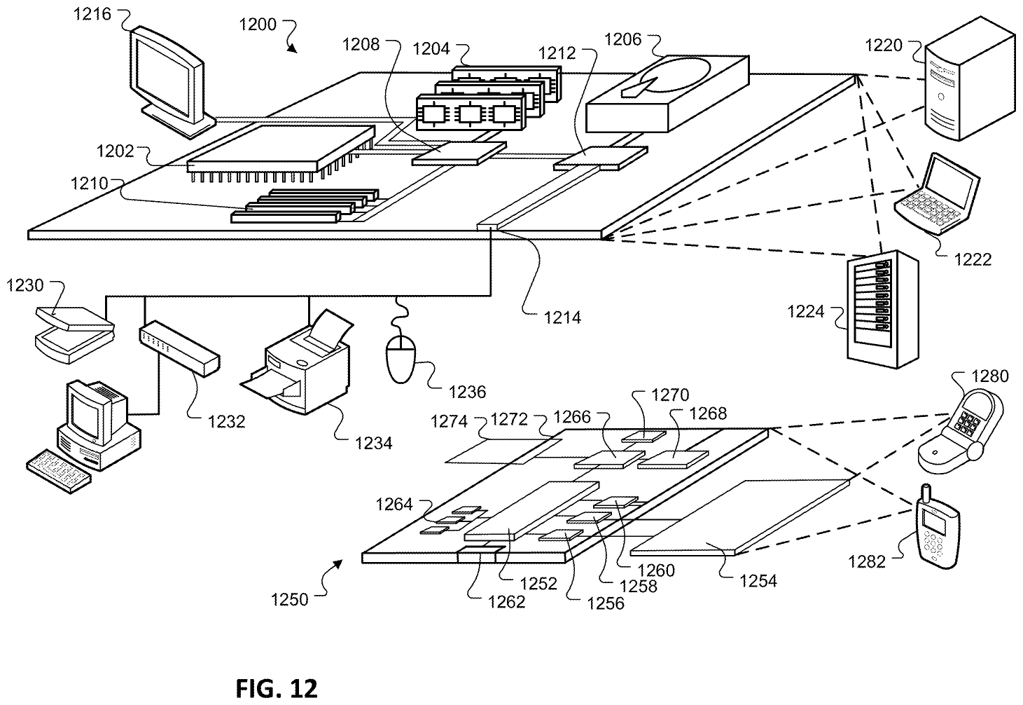

Referring now to FIG. 2, a more detailed view of step 116 of the process 100 is shown, providing additional details about how feature functions are defined. In particular, the step 116 comprises a first step 202, in which the population data 114 is received. For example, the population data 114 can be received at a computing device configured to perform the step 116. The step 116 also includes a step 204, in which the received data is processed to analyze one or more variables of interest and to define a mathematical form and one or more parameters of feature functions that correspond to the one or more variables. The processing of the received data can be performed, in some implementations, by one or more processors of the computing device configured to perform the step 116. Examples of computing devices, including mobile computing devices capable of performing the step 116 are described in further detail below, for example, in relation to FIG. 12.

Analyzing the one or more variables of interest in step 204 can include analyzing a distribution of the one or more variables in a relevant population. For example, if the eligible subjects for a study include all adults over 50 years old having a history of hypertension, then the distributions of the one or more variables of interest can be assessed, using the population data 114, for a representative sample of individuals in this target population. Graph 206 shows a cumulative distribution of one example variable—a number of visits to a healthcare facility—in an example target population. Graph 208 shows a frequency distribution (e.g., in the form of a histogram) of the same variable in the same target population. Analyzing the one or more variables of interest in step 204 can include plotting variable distributions such as those shown in graph 206 and graph 208 to determine a mathematical form and one or parameters of feature functions that correspond to the one or more variables. In some implementations, the feature functions can also be scaled based on preferences within the population (e.g., as measured using survey data, EMR data, published studies, etc.) such that variables that are relatively less impactful on patient burden are scaled to have lower estimated burdens compared to variables that are relatively more influential on patient burden.

In the example shown in FIG. 2, it is evident from the shape of the distributions shown in graphs 206, 208 that there is an exponential drop-off in the number of individuals as the number of visits increases. One interpretation of the graphs 206, 208 is that the burden of making additional visits increases exponentially with each additional visit to a healthcare facility. Therefore, a feature function corresponding to a number of visits may be defined such that an individual's estimated burden has an exponential relationship with the number of visits the individual makes to healthcare facilities. The particular parameters or coefficients for the feature function can be determined based on the specific shape of the distributions. For example, the feature function in this example may be defined as where are estimated feature parameters and x reflects the variable of interest (e.g., number of hospitalizations). If the variable of interest is number of hospitalizations, an example feature function might be:

$$burden ( x ) = \{ \begin{matrix}0 : if x < 0 , study schedule is less intensive than current care \\ \text{?} : otherwise\end{matrix}$$

$$\text{?} \text{indicates text missing or illegible when filed}$$

where x represents the difference between the number of annual hospitalizations required by a study and an individual's yearly hospitalizations. The burden value would be 1 when the individual's current yearly hospitalizations matches the study requirements exactly, 0 if the study schedule is less intense (e.g., requiring fewer hospitalizations) compared to an individual's current care, and greater than 1 if the study schedule is more intense (e.g., requiring more hospitalizations) compared to an individual's current care. If the individual has no hospitalizations at all, the burden value would assume a value of which is the maximum value.

In other examples, feature functions may be defined as having a linear form rather than an exponential form. For example, when defining the feature function for another example variable-distance from the study site-the population data 114 may include survey data that indicates that individuals experience a consistent incremental burden for every additional ten miles that they live away from the study site. Thus, in this example, a linear feature function may be more appropriate than an exponential feature function, and the feature function may be defined as where is an estimated feature parameter and x reflects the variable of interest (e.g., travel within a zip code, within a county, within a state, interstate, etc.). If the variable of interest is travel distance, an example feature function might be:

$$y ( x ) = \{ \begin{matrix}2 * x \text{?} x ∈ \{ 1 , 2 , 3 , 4 \} \\ 16 : otherwise\end{matrix}$$

$$\text{?} \text{indicates text missing or illegible when filed}$$

where x=1 when the individual needs to travel within a zip code, x=2 when the individual needs to travel within a county, x=3 when the individual needs to travel within a state, and x=4 when the individual needs to travel interstate. When data about required travel is not available, a default burden value of 16 is assumed.

In some implementations, feature functions may be defined using a machine learning-based approach. For example, one or more machine learning models can be trained on the population data 114 to approximate feature functions, predicting estimated patient burdens based on the values of one or more input variables. In some cases, the one or more machine learning models can be trained using population data 114 that includes data from previous studies. This data can include information about the participants in the previous studies, especially with respect to one or more variables of interest. This data can also include information about whether or not the study participants achieved or satisfied a particular outcome with respect to the study (e.g., enrollment in the study, retention in the study, completion of the study, adherence to a protocol of the study, etc.). The one or more machine learning models can be trained using the population data 114 to predict the likelihood of individuals achieving or satisfying a particular outcome with respect to the study (or alternatively, to predict estimated burdens, which are inversely related to the likelihood of achieving or satisfying the particular outcome) based on the values of the one or more variables of interest. In some implementations, the one or more machine learning models can employ one or more techniques including decision trees, linear regression, neural networks, multinomial logistic regression, Naive Bayes (NB), trained Gaussian NB, NB with dynamic time warping, multiple linear regression, Shannon entropy, support vector machine (SVM), one versus one support vector machine, k-means clustering, Q-learning, temporal difference (TD), neural networks, deep adversarial networks, and the like. In some implementations, a machine learning-based approach can have the advantage of flexibly estimating feature functions without having to specify a particular mathematical form for the feature functions (e.g., a linear form, an exponential form, etc.). In some implementations, the machine learning models can be implemented using an active learning approach such that real world outcomes for study subjects are compared to their predicted outcomes and fed back to the machine learning models to further train the machine learning models. Through this process, the machine learning models can continually improve in performance by collecting additional training data from every study in which the machine learning models are used.

Importantly, the feature functions defined at step 116 can be externally validated in other datasets and studies. For example, a feature function modeling the relationship between distance to a study site and an associated estimated burden may be equally applicable to a wide variety of clinical trials, and may be conveniently used for subject selection in other study protocols. In contrast to other existing methodologies for subject selection, this transportability of feature functions can therefore have the advantage of enabling direct comparisons between the estimated burdens associated with different study protocols.

Referring now to FIGS. 3A-3C, various approaches are shown for implementing step 110 of the process 100 to identify the least burdened subset of potential subjects 112 or least burdened patient profile corresponding to the subset. FIG. 3A shows a first approach 110A to implementing the step 110. Under the first approach 110A, the feature functions 106 are defined (e.g., at step 116 of the process 100) such that that ideal point has an estimated burden value of zero associated with each of the one or more variables of interest. Thus, as shown in FIG. 3A, when plotting the estimated burden values associated with each feature function (e.g., burden₁ and burden₂) on a Cartesian coordinate system, the ideal point is located at the origin. While FIG. 3A shows a 2-D graph in which the estimated burden values associated with only two feature functions are plotted, this is simply for clarity of explanation. It is recognized that the graph shown in FIG. 3A can be readily extended into n-dimensions to reflect the estimated burden values associated with n feature functions (e.g., the feature functions 106).

In the 2-D graph shown in FIG. 3A, the estimated burden values associated with a first feature function are plotted along the horizontal axis (“burden₁”), and the estimated burden values associated with a second feature function are plotted along the vertical axis (“burden₂”). Using the estimated burden values 108 for each individual in the group of potential subjects 102, a point is plotted for each individual in the 2-D graph (labeled P1-P8, respectively, to correspond to the individuals P1-P8 shown in FIG. 1). In this graphical representation, the distance (“d”) between the ideal point and each of the points P1-P8 represents an overall estimated incremental burden likely to be imposed on the individual associated with the respective point if they were to participate in a study. Therefore, the points that are closest to the ideal point represent individuals who would be least burdened by participation in the study.

In some implementations, subsets of the group of potential subjects 102 can be formed by clustering individuals based on the distance of their corresponding points to the ideal point. For example, individuals P1, P5, and P6 can comprise a first cluster of least burdened subjects 112 since their respective distances to the ideal point are all less than r₁. Individuals P2 and P3 can comprise a second cluster of slightly more burdened subjects since their respective distances to the ideal point are all greater than r₁, but less than r₂. Individuals P7 and P8 can comprise a third cluster of even more burdened subjects, since their respective distances to the ideal point are all greater than r₂, but less than r₃. And finally, individual P4 can comprise a fourth cluster of most burdened subjects since its distance to the ideal point is greater than r₄.

While the approach 110A is shown graphically in FIG. 3A, it will be readily understood by a person skilled in the art that, in some implementations, mathematical equivalents to the approach 110A can be performed without necessarily plotting points on a Cartesian coordinate system. For example, in one implementation, the estimated burden values 108 for each individual in the group of potential subjects 102 can be represented as a vector of burden values. That is, a first vector of burden values, {burden_{1,P1}, burden_{2,P1}} may be associated with individual P1; a second vector of burden values, {burden_{1,P2}, burden_{2,P2}}, may be associated with individual P2; a third vector of burden values, {burden_{1,P3}, burden_{2,P3}}, may be associated with individual P3; etc. In this implementation, calculating a magnitude of each vector is mathematically equivalent to calculating a distance from the ideal point (located at the origin) to cach of the points associated with the individuals P1-P8. Therefore, the least burdened subset 112 can be equivalently identified (and clusters of individuals can be equivalently formed) based on the vector magnitudes. Other mathematical equivalents are also envisioned.

Referring now to FIG. 3B, a second approach 110B to implementing the step 110 of the process 100 is shown. The second approach 110B is substantially similar to the first approach 110A (shown in FIG. 3A), except under the second approach 110B, the feature functions are defined (e.g. at step 116 of the process 100) such that the ideal point is not necessarily located at the origin. For example, the feature functions may be defined such that a hypothetical individual at the ideal point still has non-zero estimated burden values. Under the second approach 110B, the estimated incremental burden for each individual is not represented by the distance of each of the points P1-P8 from the origin. Rather, the estimated incremental burden imposed on each individual by participation in the study is represented by the distance of cach of the points P1-P8 from the location of the ideal point. Once again, the points that are closest to the ideal point represent individuals who would be least burdened by participation in the study.

As with the first approach 110A, it is recognized that the 2-D graph shown in FIG. 3B can be readily extended into n-dimensions to reflect the estimated burden values associated with n feature functions (e.g., the feature functions 106). Moreover, it is again recognized that mathematical equivalents to the graphical identification of the least burdened subset of potential subjects 112 can be implemented, and therefore fall within the scope of the present disclosure.

Under the approaches 110A, 110B, once a distance value (or alternatively, a vector magnitude) has been calculated for each individual in the group of potential subjects 102, various techniques can be used to identify the least burdened subset of potential subjects 112 (or a least burdened patient profile corresponding to the subset). A first technique can include ranking the individuals by their distance value (or vector magnitude) and selecting a pre-determined number or percentage of least burdened individuals (e.g., those having the lowest distance or magnitude values) for prioritization in study recruitment. A second technique can involve setting a threshold distance or magnitude value below which individuals should be prioritized for study recruitment. In some cases, the threshold distance or magnitude value can be selected based on a maximum acceptable level of estimated incremental burden to be imposed on potential subjects if they were to participate in the study. In some cases, the threshold distance or magnitude value can be selected based on analyzing the distribution of distance or magnitude values within the group of potential subjects 102. The threshold distance or magnitude value can define a least burdened patient profile such that any individuals later found to have a distance or magnitude value below the threshold distance or magnitude value can be classified as matching or satisfying the patient profile.

Referring now to FIG. 3C, a third approach 110C to implementing the step 110 of the process 100 is shown. The third approach 110C involves a direct calculation of an aggregate burden metric for each individual in the group of potential subjects 102. The aggregate burden metric is sometimes referred to herein as a “patient friction coefficient” (PFC) because when positive it represents a level of resistance that must be overcome to persuade a potential subject to participate in a study and/or satisfy one or more particular outcomes with respect to the study. When negative, the magnitude of the PFC represents the level of incentive a potential subject may have to participate in the study and/or satisfy one or more particular outcomes with respect to the study. Individuals having a higher incremental burden associated with participating in a study are likely to have higher PFC values, and individuals with lower incremental burden associated with participating in the study are likely to have lower PFC values. Individuals with a negative incremental burden (or burden reduction), are likely to benefit most in this framework from participation in the study.

The PFC for a particular individual (e.g., individuals P1-P8) can be estimated by aggregating the incremental burdens associated with multiple variables of interest. For example, in some implementations, the PFC can be computed as a weighted combination of the incremental burdens associated with multiple variables of interest and can be represented by the following equation:

$$PFC = {\sum }_{k = 1}^{n} ( {α}_{k} * {incremental\_burden}_{k} )$$

subject to:

$${\sum }_{k = 1}^{n} {α}_{k} = 1 ; {∧}_{k = 1}^{n} {α}_{k} \geq 0$$

where represents the incremental burden (or burden reduction) associated with the k^{th} variable of interest (e.g., indicators of a visit schedule, indicators of disease, age, gender, travel distance to a trial location, etc.), and represents the relative weighting of the k^{th} variable of interest (e.g., based on a relative influence of the k^{th} variable of interest on overall patient burden). As described above, the relative weighting of the one or more variables of interest can be determined based on preferences within the population (e.g., as measured using survey data, EMR data, published studies, etc.).

The incremental burden (or burden reduction) for each of the one or more variables of interest can be calculated using the feature functions 106 and the burdens associated with the ideal point (described above in relation to FIGS. 3A-3B). For example, for individual P1, the incremental burden for a first variable of interest (e.g., visit schedule) can be calculated as the difference between (i) the estimated burden value for P1 associated with the first variable of interest (“burden_{1,P1}”) and (ii) a burden value associated with the first variable of interest at the ideal point. Note that, where the ideal point is located at the origin (e.g., as in FIG. 3A), the estimated burden value associated with each of the variables of interest at the ideal point is zero, so the incremental burden for each of the variables of interest would simply be equivalent to the estimated burden values 108 calculated using the feature functions 106.

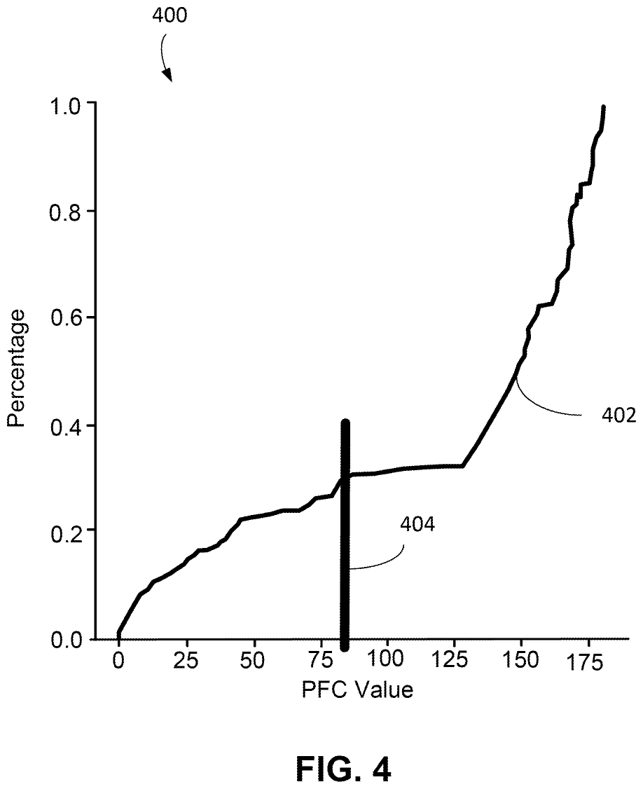

Under the approach 110C, once a PFC value has been calculated for each individual in the group of potential subjects 102, various techniques can be used to identify the least burdened subset of potential subjects 112 (or the least burdened patient profile corresponding to the least burdened subset of potential subjects 112). A first technique can include ranking the individuals by their PFC value and selecting a pre-determined number (or percentage) of least burdened individuals (e.g., those having the lowest PFC values) for prioritization in study recruitment. A second technique can involve setting a threshold PFC value below which individuals should be prioritized for study recruitment. In some cases, the threshold PFC value can be selected based on a maximum acceptable level of estimated incremental burden to be imposed on potential subjects if they were to participate in the study. In some cases, the threshold PFC value can be selected based on analyzing the distribution of PFC values within the group of potential subjects 102, as described below in relation to FIG. 4. The threshold PFC value can define a least burdened patient profile such that any individuals later found to have a PFC value below the threshold PFC value can be classified as matching or satisfying the least burdened patient profile.

FIG. 4 is a graph 400 showing an example cumulative distribution 402 of PFC values within a group of potential subjects 102. Overlaid on the graph 400, is a line 404 corresponding to a PFC threshold value of approximately 85. In this example, the PFC threshold value was set at 85 by analyzing the cumulative distribution 402. More specifically, as shown by the cumulative distribution 402, the number of cumulative individuals steadily increased with increasing PFC values until reaching a plateau between PFC values of approximately 85 and 130, and then increased rapidly again above PFC values of 130. The PFC threshold value was therefore set at 85 in this example since it represents a natural cluster of individuals in the group of potential subjects 102. Setting the PFC threshold at a higher value would not result in the inclusion of many more individuals in the least burdened subset 112 until the PFC threshold exceeds 130. However, beyond a PFC threshold value of 130, any additional individuals prioritized for recruitment would have relatively high PFC values and therefore may result in wasted time, money, and effort to recruit.

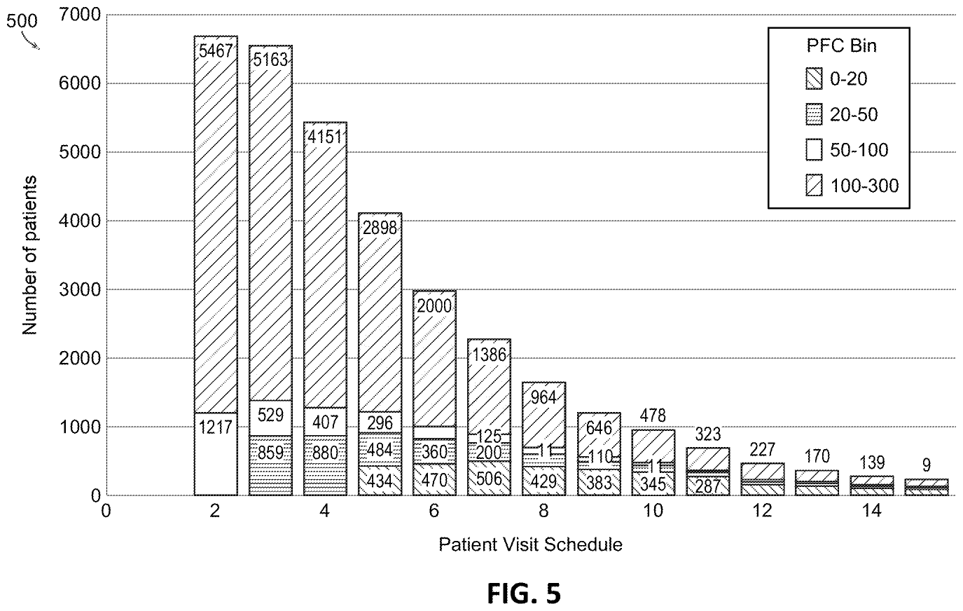

Referring now to FIGS. 5-8, techniques are described for verifying the utility of feature functions once they are defined (e.g., at step 116 of the process 100 shown in FIG. 1). One technique for verifying the utility of a feature function is to analyze the distribution(s) of the one or more associated variables of interest and assess whether there are substantial changes in the breakdown of PFC values along the distribution. For example, FIG. 5 is a graph 500 showing a distribution of visit schedules for a group of potential subjects 102. Each bar within the graph 500 is subdivided into bins defined by a range of PFC values (0-20, 20-50, 50-100, and 100-300). If visit schedule is an influential variable affecting the incremental burden imposed on potential subjects, one might expect to see lower PFC values associated with individuals who currently make a higher number of visits to healthcare facilities compared to those who currently make fewer visits to healthcare facilities. This is because individuals who already make many hospital visits may be less burdened by the number of visits required by a study protocol. Indeed, as demonstrated in the graph 500, as individuals make increasing visits to healthcare facilities (e.g., moving right along the x-axis), an increasing proportion of those individuals have relatively low PFC values (e.g., as demonstrated by the changing breakdown of PFC values within the individual bars). Therefore, the feature function associated with the “visit schedule” variable can be considered verified as having utility for discriminating between individuals with varying levels of estimated incremental burdens (or burden reductions).

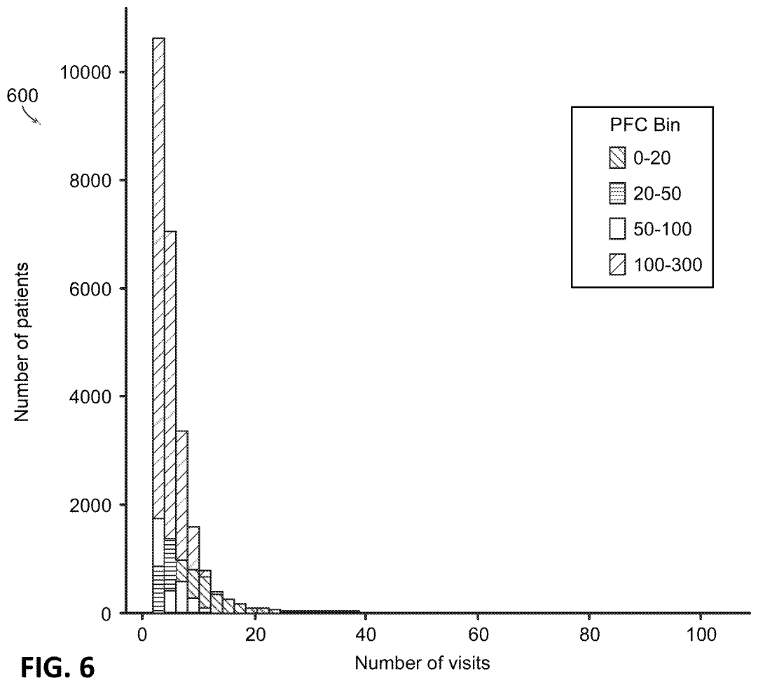

Another technique for verifying the utility of a feature function is shown in FIG. 6. Once again, the “visit schedule” variable (also referred to as “number of visits”) is used as an example. The graph 600 shows the distribution of the “visit schedule” variable in this eligible population, but includes separate histograms for the individuals falling into each bin of PFC values (0-20, 20-50, 50-100, 100-200). As shown in the graph 600, the histograms associated with individuals having lower PFC values were more heavily weighted toward higher numbers of visits, while the histograms associated with individuals having higher PFC values were more heavily weighted toward lower numbers of visits. Thus, this technique also confirms the utility of the feature function associated with the “visit schedule” variable for discriminating between individuals with varying levels of estimated incremental burdens (or burden reductions), at least for some patient populations or diseases. For a different patient population or disease this variable might have different utility (or no utility at all).

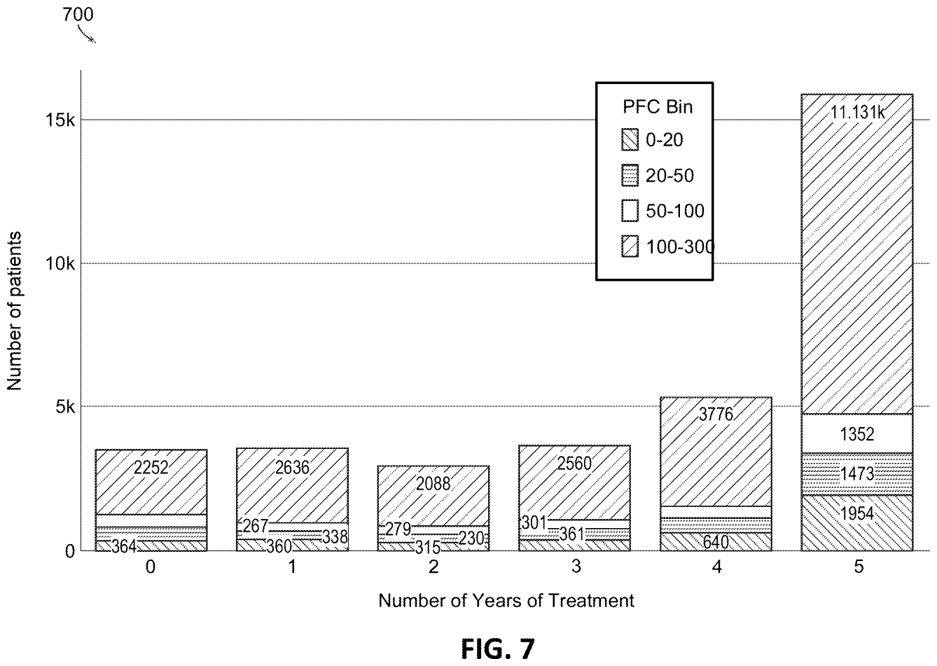

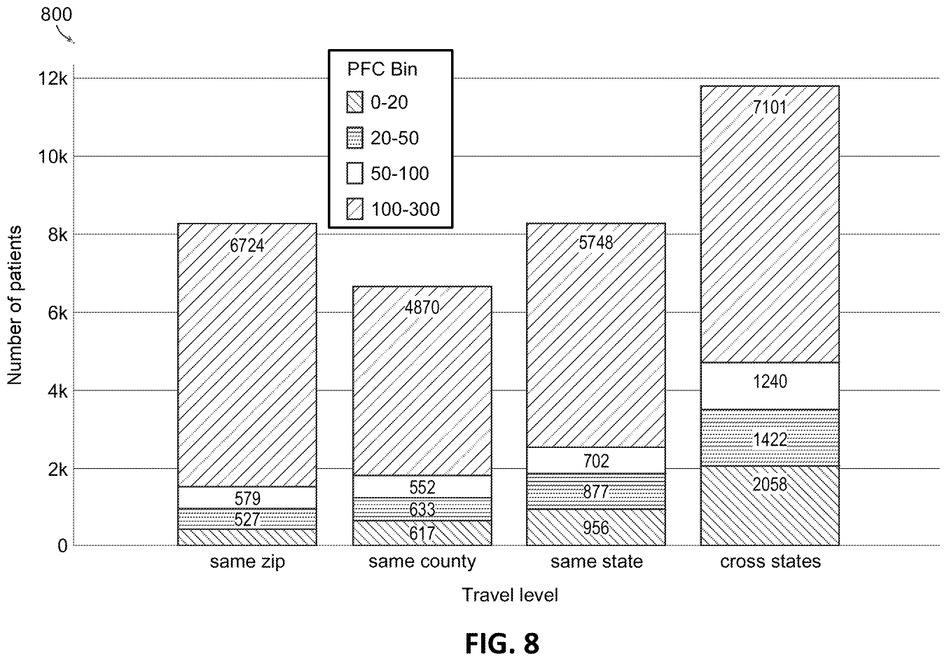

FIGS. 7-8 provide examples where verifying the utility of feature functions reveals that the feature functions have more limited utility for discriminating between individuals eligible for this protocol with varying levels of estimated incremental burdens (or burden reductions). For example, FIG. 7 is a graph 700 showing a distribution of a “number of years of treatment” variable for a group of potential subjects 102. Each bar within the graph 700 is subdivided into bins defined by a range of PFC values (0-20, 20-50, 50-100, and 100-300). If number of years of treatment is an influential variable affecting the incremental burden imposed on potential subjects, one might expect to see lower PFC values associated with individuals who have been treated for many years since they are likely to have a more severe disease. However, as demonstrated in the graph 700, as number of years of treatment increases (e.g., moving right along the x-axis), there is no substantial change in the proportional breakdown of PFC values within the individual bars. Therefore, the feature function associated with the “number of years of treatment” variable can be considered to have limited value for discriminating between individuals with varying levels of estimated incremental burdens (or burden reductions). Consequently, when determining the least burdened subset of potential subjects 112, the feature function associated with the “number of years of treatment” variable can, in some implementations for some patient populations or diseases, be safely excluded since it is likely to have little impact on the subject selection process.

FIG. 8 is a graph 800 showing a distribution of a “travel level” variable for a group of potential subjects 102. Each bar within the graph 800 is subdivided into bins defined by a range of PFC values (0-20, 20-50, 50-100, and 100-300). If travel level is an influential variable affecting the incremental burden imposed on potential subjects, one might expect to see lower PFC values associated with individuals who have to travel the shortest distance since they are likely to be the least burdened by traveling to the study site. However, as demonstrated in the graph 800, as travel level increases (e.g., moving right along the x-axis), there is no substantial change in the proportional breakdown of PFC values within the individual bars. Therefore, the feature function associated with the “travel level” variable can be considered to have limited value for discriminating between individuals with varying levels of estimated incremental burdens (or burden reductions). Consequently, when determining the least burdened subset of potential subjects 112, the feature function associated with the “travel level” variable can, in some implementations for some patient populations or diseases, be safely excluded since it is likely to have little impact on the subject selection process.

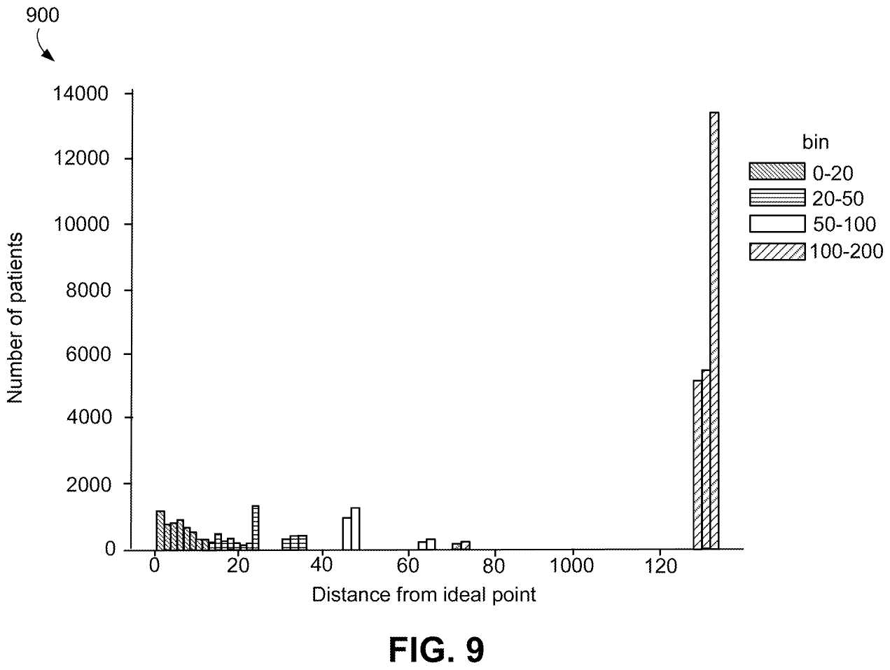

Referring now to FIG. 9, one can also verify that the overall PFC calculation approach (described in relation to FIG. 3C) and the distance segmentation approach (described in relation to FIG. 3A) yield similar rankings for the group of potential subjects 102 based on their estimated incremental burden (or estimated burden reduction) levels. The graph 900 shows the distribution of the “distance from ideal point” values (represented by “d” in FIG. 3A) in the group of potential subjects 102, but includes separate histograms for the individuals falling into different bins of PFC values (0-20, 20-50, 50-100, and 100-200). As shown in the graph 900, there was good agreement between the PFC calculation approach and the distance segmentation approach, with the same individuals being identified as the least burdened individuals under both approaches (e.g., having both low PFC values and low distance from the ideal point). There was also good agreement in identifying the most burdened individuals under both approaches (e.g., having both high PFC values and high distance from the ideal point).

While the validation results shown in FIG. 9 represent the results of an internal validation approach, the technology described in this document can also be externally validated. For example, after ranking participants in a study based on their respective estimated incremental burden (or burden reduction) levels, one can observe whether or not the individuals identified as belonging to the least burdened subset of potential subjects 112 actually achieve or satisfy a particular outcome with respect to the study at higher rates compared to the other subjects. This methodology can be repeated across a number of different studies (e.g., clinical trials) to externally validate the techniques described herein.

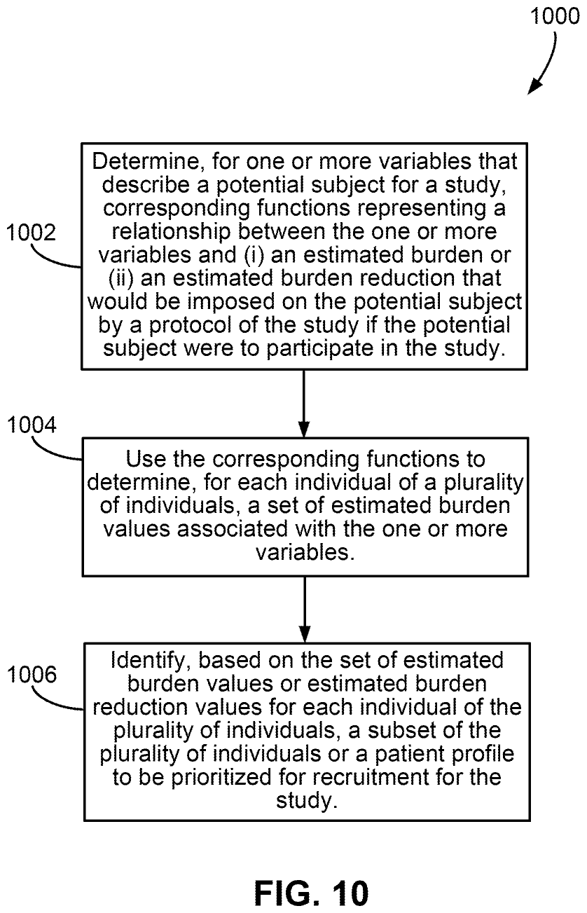

FIG. 10 illustrates an example process 1000 for identifying a subset of the plurality of individuals or a patient profile to be prioritized for recruitment for a study. In some implementations, operations of the process 1000 can be executed by a computing device or mobile computing device such as those described below in relation to FIG. 12.

Operations of the process 1000 include determining, for one or more variables that describe a potential subject for a study, corresponding functions representing a relationship between the one or more variables and (i) an estimated burden or (ii) an estimated burden reduction that would be imposed on the potential subject by a protocol of the study if the potential subject were to participate in the study (1002). The one or more variables can include at least one of a visit schedule, an indicator of disease, an age, a gender, and a travel distance to a trial location. For each individual of the plurality of individuals, the subject-specific values for the one or more variables can be determined, at least in part, based on questionnaire data or electronic medical records (EMR) data. For each individual of the plurality of individuals, at least some of the subject-specific values for the one or more variables can represent historical values for the individual and/or predicted future values for the individual. In some implementations, the estimated burden can be inversely related to a likelihood that the potential subject satisfies a particular outcome with respect to the study (e.g., enrollment in the study, retention in the study, completion of the study, or adherence to a protocol of the study). In some implementations, the corresponding functions can be convex and monotonically increasing. Determining the corresponding functions can include determining a mathematical form of each of the corresponding functions based on distributions of the one or more variables in a population and/or determining one or more parameters of the corresponding functions based on at least one of questionnaire data, electronic medical records (EMR) data, or subject matter expertise. In some implementations, determining the corresponding functions can include defining at least some of the corresponding functions such that the estimated burden is zero when the subject-specific values for the one or more variables absent participation of the potential subject in the study match values for the one or more variables specified by the protocol of the study.

Operations of the process 1000 also include using the corresponding functions to determine, for each individual of a plurality of individuals, a set of estimated burden values associated with the one or more variables (1004).

Operations of the process 1000 also include identifying, based on the set of estimated burden values or estimated burden reduction values for each individual of the plurality of individuals, a subset of the plurality of individuals or a patient profile to be prioritized for recruitment for the study (1006). For example, the subset of the plurality of individuals can correspond to the least burdened subset 112 of the group potential subjects 102 described in relation to FIG. 1. Identifying the subset of the plurality of individuals or a patient profile to be prioritized for recruitment for the study can include (i) computing, for each individual of the plurality of individuals, an aggregate metric (e.g., a PFC value) representative of the set of estimated burden values, and (ii) identifying whether each individual of the plurality of individuals is below, above, or equal to a threshold value of the aggregate metric. In some implementations, the aggregate metric can be externally validated. The aggregate metric can be indicative of at least one of (i) a magnitude of a vector defined, at least in part, by the set of estimated burdens values or estimated burden reduction values (e.g., as in the approaches 110A, 110B described in relation to FIGS. 3A-3B), or (ii) a weighted combination of the set of estimated burden values or estimated burden reduction values (e.g., as in the approach 110C described in relation to FIG. 3C). One or more weights of the weighted combination can be determined based on survey data reflective of one or more preferences of a population with respect to the one or more variables. In some implementations, identifying the subset of the plurality of individuals or the patient profile to be prioritized for recruitment for the study can include (i) computing, for each individual of the plurality of individuals, an aggregate metric (e.g., a PFC value) representative of the set of estimated burden values or estimated burden reduction values, and (ii) identifying a specified number of the plurality of individuals or a specified percentage of the plurality of individuals based on a ranking of the plurality of individuals according to the aggregate metric.

Additional operations of the process 1000 can include the following. In some implementations, the process 1000 can include recruiting the identified individuals or individuals that fit the patient profile for the study and/or treating at least a portion of the identified individuals or individuals that fit the patient profile in accordance with the protocol of the study. In some implementations, the process 1000 can include (i) determining that the identified subset of the plurality of individuals to be prioritized for recruitment for the study is below a threshold size, and (ii) altering the protocol of the study with respect to at least some of the one or more variables to increase a size of the identified subset of the plurality of individuals to be prioritized for recruitment for the study. In some implementations, the process 1000 can include comparing the protocol of the study with at least one other study protocol, wherein comparing the protocol of the study with the at least one other study protocol can include comparing the set of estimated burden values or estimated burden reduction values for each individual of the plurality of individuals with other burden-related calculations for the plurality of individuals, wherein the other burden-related calculations are associated with the at least one other study protocol.

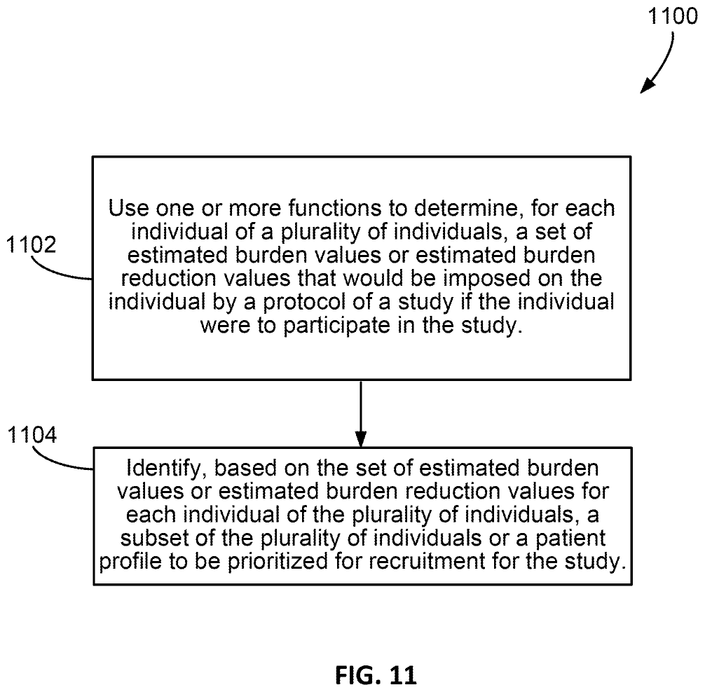

FIG. 11 illustrates another example process 1100 for identifying a subset of the plurality of individuals or a patient profile to be prioritized for recruitment for a study. In some implementations, operations of the process 1100 can be executed by a computing device or mobile computing device such as those described below in relation to FIG. 12.

Operations of the process 1100 include using one or more functions to determine, for each individual of a plurality of individuals, a set of estimated burden values or estimated burden reduction values that would be imposed on the individual by a protocol of a study if the individual were to participate in the study (1102). The one or more functions can correspond to the feature functions described above and can be defined in accordance with the step 116 of the process 100 described in relation to FIG. 1. In some implementations, the one or more functions can be pre-defined by a third party and/or can be defined using one or more other processes other than those described above in relation to step 116. In some implementations, the one or more functions can be convex and monotonically increasing. The estimated burden values can be inversely related to a likelihood that the potential subject satisfies a particular outcome with respect to the study (e.g., enrollment in the study, retention in the study, completion of the study, or adherence to a protocol of the study).

Operations of the process 1100 also include identifying, based on the set of estimated burden values or estimated burden reduction values for each individual of the plurality of individuals, a subset of the plurality of individuals or a patient profile to be prioritized for recruitment for the study (1104). For example, the subset of the plurality of individuals can correspond to the least burdened subset 112 of the group potential subjects 102 described in relation to FIG. 1. Identifying the subset of the plurality of individuals or the patient profile to be prioritized for recruitment for the study can include (i) computing, for each individual of the plurality of individuals, an aggregate metric (e.g., a PFC value) representative of the set of estimated burden values, and (ii) identifying whether each individual of the plurality of individuals is below, above, or equal to a threshold value of the aggregate metric. In some implementations, the aggregate metric can be externally validated. The aggregate metric can be indicative of at least one of (i) a magnitude of a vector defined, at least in part, by the set of estimated burdens values or estimated burden reduction values (e.g., as in the approaches 110A, 110B described in relation to FIGS. 3A-3B), or (ii) a weighted combination of the set of estimated burden values or estimated burden reduction values (e.g., as in the approach 110C described in relation to FIG. 3C). One or more weights of the weighted combination can be determined based on survey data reflective of one or more preferences of a population with respect to the one or more variables. In some implementations, identifying the subset of the plurality of individuals or a patient profile to be prioritized for recruitment for the study can include (i) computing, for each individual of the plurality of individuals, an aggregate metric (e.g., a PFC value) representative of the set of estimated burden values or estimated burden reduction values, and (ii) identifying a specified number of the plurality of individuals or a specified percentage of the plurality of individuals based on a ranking of the plurality of individuals according to the aggregate metric.

Additional operations of the process 1100 can include the following. In some implementations, the process 1100 can include recruiting the identified individuals or individuals that fit the patient profile for the study and/or treating at least a portion of the identified individuals or individuals that fit the patient profile in accordance with the protocol of the study. In some implementations, the process 1100 can include (i) determining that the identified subset of the plurality of individuals to be prioritized for recruitment for the study is below a threshold size, and (ii) altering the protocol of the study with respect to at least some of the one or more variables to increase a size of the identified subset of the plurality of individuals to be prioritized for recruitment for the study. In some implementations, the process 1100 can include comparing the protocol of the study with at least one other study protocol, wherein comparing the protocol of the study with the at least one other study protocol can include comparing the set of estimated burden values or estimated burden reduction values for each individual of the plurality of individuals with other burden-related calculations for the plurality of individuals, wherein the other burden-related calculations are associated with the at least one other study protocol.

FIG. 12 shows an example of a computing device 1200 and a mobile computing device 1250 that are employed to execute implementations of the present disclosure. For example, the computing device 1200 and/or the mobile computing device can be employed to execute various steps of the process 100 such as defining feature functions (step 116), calculating estimated burden values 108 using the feature functions 106 and data 104, and identifying the least burdened subset of potential subjects (step 110). The computing device 1200 is intended to represent various forms of digital computers, such as laptops, desktops, workstations, personal digital assistants, servers, blade servers, mainframes, and other appropriate computers. The mobile computing device 1250 is intended to represent various forms of mobile devices, such as personal digital assistants, cellular telephones, smart-phones, AR devices, and other similar computing devices. The components shown here, their connections and relationships, and their functions, are meant to be examples only, and are not meant to be limiting.

The computing device 1200 includes a processor 1202, a memory 1204, a storage device 1206, a high-speed interface 1208, and a low-speed interface 1212. In some implementations, the high-speed interface 1208 connects to the memory 1204 and multiple high-speed expansion ports 1210. In some implementations, the low-speed interface 1212 connects to a low-speed expansion port 1214 and the storage device 1204. Each of the processor 1202, the memory 1204, the storage device 1206, the high-speed interface 1208, the high-speed expansion ports 1210, and the low-speed interface 1212, are interconnected using various buses, and may be mounted on a common motherboard or in other manners as appropriate. The processor 1202 can process instructions for execution within the computing device 1200, including instructions stored in the memory 1204 and/or on the storage device 1206 to display graphical information for a graphical user interface (GUI) on an external input/output device, such as a display 1216 coupled to the high-speed interface 1208. In other implementations, multiple processors and/or multiple buses may be used, as appropriate, along with multiple memories and types of memory. In addition, multiple computing devices may be connected, with each device providing portions of the necessary operations (e.g., as a server bank, a group of blade servers, or a multi-processor system).

The memory 1204 stores information within the computing device 1200. In some implementations, the memory 1204 is a volatile memory unit or units. In some implementations, the memory 1204 is a non-volatile memory unit or units. The memory 1204 may also be another form of a computer-readable medium, such as a magnetic or optical disk.

The storage device 1206 is capable of providing mass storage for the computing device 1200. In some implementations, the storage device 1206 may be or include a computer-readable medium, such as a floppy disk device, a hard disk device, an optical disk device, a tape device, a flash memory, or other similar solid-state memory device, or an array of devices, including devices in a storage area network or other configurations. Instructions can be stored in an information carrier. The instructions, when executed by one or more processing devices, such as processor 1202, perform one or more methods, such as those described above. The instructions can also be stored by one or more storage devices, such as computer-readable or machine-readable mediums, such as the memory 1204, the storage device 1206, or memory on the processor 1202.

The high-speed interface 1208 manages bandwidth-intensive operations for the computing device 1200, while the low-speed interface 1212 manages lower bandwidth-intensive operations. Such allocation of functions is an example only. In some implementations, the high-speed interface 1208 is coupled to the memory 1204, the display 1216 (e.g., through a graphics processor or accelerator), and to the high-speed expansion ports 1210, which may accept various expansion cards. In the implementation, the low-speed interface 1212 is coupled to the storage device 1206 and the low-speed expansion port 1214. The low-speed expansion port 1214, which may include various communication ports (e.g., Universal Serial Bus (USB), Bluetooth, Ethernet, wireless Ethernet) may be coupled to one or more input/output devices. Such input/output devices may include a scanner, a printing device, or a keyboard or mouse. The input/output devices may also be coupled to the low-speed expansion port 1214 through a network adapter. Such network input/output devices may include, for example, a switch or router.

The computing device 1200 may be implemented in a number of different forms, as shown in FIG. 12. For example, it may be implemented as a standard server 1220, or multiple times in a group of such servers. In addition, it may be implemented in a personal computer such as a laptop computer 1222. It may also be implemented as part of a rack server system 1224. Alternatively, components from the computing device 1200 may be combined with other components in a mobile device, such as a mobile computing device 1250. Each of such devices may contain one or more of the computing device 1200 and the mobile computing device 1250, and an entire system may be made up of multiple computing devices communicating with each other.

The mobile computing device 1250 includes a processor 1252; a memory 1264; an input/output device, such as a display 1254; a communication interface 1266; and a transceiver 1268; among other components. The mobile computing device 1250 may also be provided with a storage device, such as a micro-drive or other device, to provide additional storage. Each of the processor 1252, the memory 1264, the display 1254, the communication interface 1266, and the transceiver 1268, are interconnected using various buses, and several of the components may be mounted on a common motherboard or in other manners as appropriate. In some implementations, the mobile computing device 1250 may include a camera device(s).

The processor 1252 can execute instructions within the mobile computing device 1250, including instructions stored in the memory 1264. The processor 1252 may be implemented as a chipset of chips that include separate and multiple analog and digital processors. For example, the processor 1252 may be a Complex Instruction Set Computers (CISC) processor, a Reduced Instruction Set Computer (RISC) processor, or a Minimal Instruction Set Computer (MISC) processor. The processor 1252 may provide, for example, for coordination of the other components of the mobile computing device 1250, such as control of user interfaces (UIs), applications run by the mobile computing device 1250, and/or wireless communication by the mobile computing device 1250.

The processor 1252 may communicate with a user through a control interface 1258 and a display interface 1256 coupled to the display 1254. The display 1254 may be, for example, a Thin-Film-Transistor Liquid Crystal Display (TFT) display, an Organic Light Emitting Diode (OLED) display, or other appropriate display technology. The display interface 1256 may include appropriate circuitry for driving the display 1254 to present graphical and other information to a user. The control interface 1258 may receive commands from a user and convert them for submission to the processor 1252. In addition, an external interface 1262 may provide communication with the processor 1252, so as to enable near area communication of the mobile computing device 1250 with other devices. The external interface 1262 may provide, for example, for wired communication in some implementations, or for wireless communication in other implementations, and multiple interfaces may also be used.

The memory 1264 stores information within the mobile computing device 1250. The memory 1264 can be implemented as one or more of a computer-readable medium or media, a volatile memory unit or units, or a non-volatile memory unit or units. An expansion memory 1274 may also be provided and connected to the mobile computing device 1250 through an expansion interface 1272, which may include, for example, a Single in Line Memory Module (SIMM) card interface. The expansion memory 1274 may provide extra storage space for the mobile computing device 1250, or may also store applications or other information for the mobile computing device 1250. Specifically, the expansion memory 1274 may include instructions to carry out or supplement the processes described above, and may include secure information also. Thus, for example, the expansion memory 1274 may be provided as a security module for the mobile computing device 1250, and may be programmed with instructions that permit secure use of the mobile computing device 1250. In addition, secure applications may be provided via the SIMM cards, along with additional information, such as placing identifying information on the SIMM card in a non-hackable manner.

The memory may include, for example, flash memory and/or non-volatile random access memory (NVRAM), as discussed below. In some implementations, instructions are stored in an information carrier. The instructions, when executed by one or more processing devices, such as processor 1252, perform one or more methods, such as those described above. The instructions can also be stored by one or more storage devices, such as one or more computer-readable or machine-readable mediums, such as the memory 1264, the expansion memory 1274, or memory on the processor 1252. In some implementations, the instructions can be received in a propagated signal, such as, over the transceiver 1268 or the external interface 1262.

The mobile computing device 1250 may communicate wirelessly through the communication interface 1266, which may include digital signal processing circuitry where necessary. The communication interface 1266 may provide for communications under various modes or protocols, such as Global System for Mobile communications (GSM) voice calls, Short Message Service (SMS), Enhanced Messaging Service (EMS), Multimedia Messaging Service (MMS) messaging, code division multiple access (CDMA), time division multiple access (TDMA), Personal Digital Cellular (PDC), Wideband Code Division Multiple Access (WCDMA), CDMA2000, General Packet Radio Service (GPRS). Such communication may occur, for example, through the transceiver 1268 using a radio frequency. In addition, short-range communication, such as using a Bluetooth or Wi-Fi, may occur. In addition, a Global Positioning System (GPS) receiver module 1270 may provide additional navigation-and location-related wireless data to the mobile computing device 1250, which may be used as appropriate by applications running on the mobile computing device 1250.

The mobile computing device 1250 may also communicate audibly using an audio codec 1260, which may receive spoken information from a user and convert it to usable digital information. The audio codec 1260 may likewise generate audible sound for a user, such as through a speaker, e.g., in a handset of the mobile computing device 1250. Such sound may include sound from voice telephone calls, may include recorded sound (e.g., voice messages, music files, etc.) and may also include sound generated by applications operating on the mobile computing device 1250.

The mobile computing device 1250 may be implemented in a number of different forms, as shown in FIG. 12. For example, it may be implemented a phone device 1280, a personal digital assistant 1282, and a tablet device (not shown). The mobile computing device 1250 may also be implemented as a component of a smart-phone, AR device, or other similar mobile device.

Computing device 1200 and/or 1250 can also include USB flash drives. The USB flash drives may store operating systems and other applications. The USB flash drives can include input/output components, such as a wireless transmitter or USB connector that may be inserted into a USB port of another computing device.

Other embodiments and applications not specifically described herein are also within the scope of the following claims. Elements of different implementations described herein may be combined to form other embodiments not specifically set forth above. Elements may be left out of the structures described herein without adversely affecting their operation. Furthermore, various separate elements may be combined into one or more individual elements to perform the functions described herein.

What is claimed is:

1. A method comprising:
determining, for one or more variables that describe a potential subject for a study, corresponding functions representing a relationship between the one or more variables and (i) an estimated burden or (ii) an estimated burden reduction that would be imposed on the potential subject by a protocol of the study if the potential subject were to participate in the study,
wherein the estimated burden or the estimated burden reduction is dependent on subject-specific values for the one or more variables absent participation of the potential subject in the study;
using the corresponding functions to determine, for each individual of a plurality of individuals, a set of estimated burden values or estimated burden reduction values associated with the one or more variables; and
identifying, based on the set of estimated burden values or estimated burden reduction values for each individual of the plurality of individuals, a subset of the plurality of individuals or a patient profile to be prioritized for recruitment for the study.

2. The method of claim 1, wherein the one or more variables include at least one of a visit schedule, an indicator of disease, an age, a gender, and a travel distance to a trial location.

3. The method of claim 1, wherein, for each individual of the plurality of individuals, the subject-specific values for the one or more variables are determined, at least in part, based on questionnaire data or electronic medical records (EMR) data.

4. The method of claim 1, wherein, for each individual of the plurality of individuals, at least some of the subject-specific values for the one or more variables represent historical values for the individual.

5. The method of claim 1, wherein, for each individual of the plurality of individuals, at least some of the subject-specific values for the one or more variables represent predicted future values for the individual.

6. The method of claim 1, wherein the estimated burden is inversely related to a likelihood that the potential subject satisfies a particular outcome with respect to the study.

7. The method of claim 6, wherein the particular outcome comprises at least one of enrollment in the study, retention in the study, completion of the study, or adherence to a protocol of the study.

8. The method of claim 1, wherein the corresponding functions are convex and monotonically increasing.

9. The method of claim 1, wherein determining the corresponding functions comprises determining a mathematical form of each of the corresponding functions based on distributions of the one or more variables in a population.

10. The method of claim 1, wherein determining the corresponding functions comprises determining one or more parameters of the corresponding functions based on at least one of questionnaire data, electronic medical records (EMR) data, or subject matter expertise.

11. The method of claim 1, wherein determining the corresponding functions comprises defining at least some of the corresponding functions such that the estimated burden is zero when the subject-specific values for the one or more variables absent participation of the potential subject in the study match values for the one or more variables specified by the protocol of the study.

12. The method of claim 1, wherein identifying the subset of the plurality of individuals or the patent profile to be prioritized for recruitment for the study comprises:
computing, for each individual of the plurality of individuals, an aggregate metric representative of the set of estimated burden values; and
identifying whether each individual of the plurality of individuals is below, above, or equal to a threshold value of the aggregate metric.

13. The method of claim 12, wherein the aggregate metric is externally validated.

14. The method of claim 12, wherein the aggregate metric is indicative of at least one of (i) a magnitude of a vector defined, at least in part, by the set of estimated burdens values or estimated burden reduction values, or (ii) a weighted combination of the set of estimated burden values or estimated burden reduction values.

15. The method of claim 14, wherein one or more weights of the weighted combination are determined based on survey data reflective of one or more preferences of a population with respect to the one or more variables.

16. The method of claim 1, wherein identifying the subset of the plurality of individuals or the patient profile to be prioritized for recruitment for the study comprises:
computing, for each individual of the plurality of individuals, an aggregate metric representative of the set of estimated burden values or estimated burden reduction values; and
identifying a specified number of the plurality of individuals or a specified percentage of the plurality of individuals based on a ranking of the plurality of individuals according to the aggregate metric.

17. The method of claim 1, further comprising, recruiting the identified individuals, or individuals that fit the patient profile, for the study.

18. The method of claim 1, further comprising treating at least a portion of the identified individuals or individuals that fit the patient profile in accordance with the protocol of the study.

19. The method of claim 1, further comprising:
determining that the identified subset of the plurality of individuals to be prioritized for recruitment for the study is below a threshold size; and
altering the protocol of the study with respect to at least some of the one or more variables to increase a size of the identified subset of the plurality of individuals to be prioritized for recruitment for the study. 20 The method of claim 1, further comprising comparing the protocol of the study with at least one other study protocol, wherein comparing the protocol of the study with the at least one other study protocol comprises:
comparing the set of estimated burden values or estimated burden reduction values for each individual of the plurality of individuals with other burden-related calculations for the plurality of individuals, wherein the other burden-related calculations are associated with the at least one other study protocol.

21. A system comprising:
a computing device comprising:
a memory configured to store instructions; and
a processor configured to execute the instructions to perform operations comprising:
determining, for one or more variables that describe a potential subject for a study, corresponding functions representing a relationship between the one or more variables and (i) an estimated burden or (ii) an estimated burden reduction that would be imposed on the potential subject by a protocol of the study if the potential subject were to participate in the study,
wherein the estimated burden or the estimated burden reduction is dependent on subject-specific values for the one or more variables absent participation of the potential subject in the study;
using the corresponding functions to determine, for each individual of a plurality of individuals, a set of estimated burden values or estimated burden reduction values associated with the one or more variables; and
identifying, based on the set of estimated burden values or estimated burden reduction values for each individual of the plurality of individuals, a subset of the plurality of individuals or a patient profile to be prioritized for recruitment for the study.

22. One or more machine-readable storage devices having encoded thereon computer readable instructions for causing one or more processing devices to perform operations comprising:
determining, for one or more variables that describe a potential subject for a study, corresponding functions representing a relationship between the one or more variables and (i) an estimated burden or (ii) an estimated burden reduction that would be imposed on the potential subject by a protocol of the study if the potential subject were to participate in the study, wherein the estimated burden or the estimated burden reduction is dependent on subject-specific values for the one or more variables absent participation of the potential subject in the study;
using the corresponding functions to determine, for each individual of a plurality of individuals, a set of estimated burden values or estimated burden reduction values associated with the one or more variables; and
identifying, based on the set of estimated burden values or estimated burden reduction values for each individual of the plurality of individuals, a subset of the plurality of individuals or a patient profile to be prioritized for recruitment for the study.

23. A method comprising:
using one or more functions to determine, for each individual of a plurality of individuals, a set of estimated burden values or estimated burden reduction values that would be imposed on the individual by a protocol of a study if the individual were to participate in the study,
wherein the one or more functions correspond to one or more variables that describe the individual, and wherein the set of estimated burden values or estimated burden reduction values is dependent on individual-specific values for the one or more variables absent participation of the individual in the study; and
identifying, based on the set of estimated burden values or estimated burden reduction values for each individual of the plurality of individuals, a subset of the plurality of individuals or a patient profile to be prioritized for recruitment for the study.
